(19) United States
(12) Patent Application Publication
(10) Pub. No.: US 2013/0064779 A1
(43) Pub. Date: Mar. 14, 2013

(54) ORAL CARE COMPOSITIONS RESISTANT TO MICROBIAL GROWTH

(71) Applicants: Odete Tieko Yamane, Sao Paulo (BR); Fernanda Cristina Geraldo Correa, Sao Paulo (BR); Sandra Pereira Ramos, Sao Paulo (BR); Katrin Chaves Da Costa, Sao Paulo (BR); Enzo Toshio Utima, Sao Paulo (BR); Erico Vieira Prat, Sao Paulo (BR); Michael Prencipe, West Windsor, NJ (US); Richard Scott Robinson, Belle Mead, NJ (US)

(72) Inventors: Odete Tieko Yamane, Sao Paulo (BR); Fernanda Cristina Geraldo Correa, Sao Paulo (BR); Sandra Pereira Ramos, Sao Paulo (BR); Katrin Chaves Da Costa, Sao Paulo (BR); Enzo Toshio Utima, Sao Paulo (BR); Erico Vieira Prat, Sao Paulo (BR); Michael Prencipe, West Windsor, NJ (US); Richard Scott Robinson, Belle Mead, NJ (US)

(73) Assignee: Colgate-Palmolive Company, New York, NY (US)

(21) Appl. No.: 13/699,012

(22) PCT Filed: Jun. 1, 2010
(86) PCT No.: PCT/US10/36891

§ 371 Date: Nov. 20, 2012

(51) Int. Cl. A61K 8/34; A61Q 11/00; A61K 8/44; A61K 8/365; A61K 8/36

(52) U.S. Cl. 424/54; 424/49; 424/57; 424/55

(57) ABSTRACT

Described herein are compositions which prevent the growth of bacteria resistant to high salt concentrations, methods of preparing the same, and methods of using the same.

Microbial contamination of oral care products poses a serious threat to the health of consumers. Thus, there is a need for oral care products that provide consistent and reproducible resistance to bacterial growth, while maintaining their efficacy and consumer acceptability.

### SUMMARY

Some embodiments of the present invention provide oral care compositions comprising: an alkaline earth metal salt; benzyl alcohol; one or more precipitation agents; and one or more alkaline earth metal ion scavenging agents.

Other embodiments provide oral care compositions comprising: from about 35% to about 45%, by weight, of an alkaline earth metal salt; about 0.5%, by weight, of a precipitation agent; about 0.5%, by weight, of a alkaline earth metal ion scavenging agent; and about 0.3%, by weight, benzyl alcohol.

Yet further embodiments provide oral care compositions comprising: an alkaline earth metal salt; and a bacteriostatic system, wherein said bacteriostatic system comprises one or more precipitation agents; one or more alkaline earth metal ion scavenging agents; and benzyl alcohol.

In some embodiments, the alkaline earth metal salt is a calcium salt or a strontium salt.

In some embodiments, the compositions described herein prevent or inhibit the growth of bacteria resistant to high salt concentrations.

Some embodiments provide methods of preventing, treating or inhibiting a disease, disorder or condition of the oral cavity comprising contacting an oral cavity surface with any of the compositions described herein.

### BRIEF DESCRIPTION OF THE DRAWING

FIG. 1 provides contour plots which demonstrate the effect of the components of the bacteriostatic system on the micro-robustness of the compositions described herein.

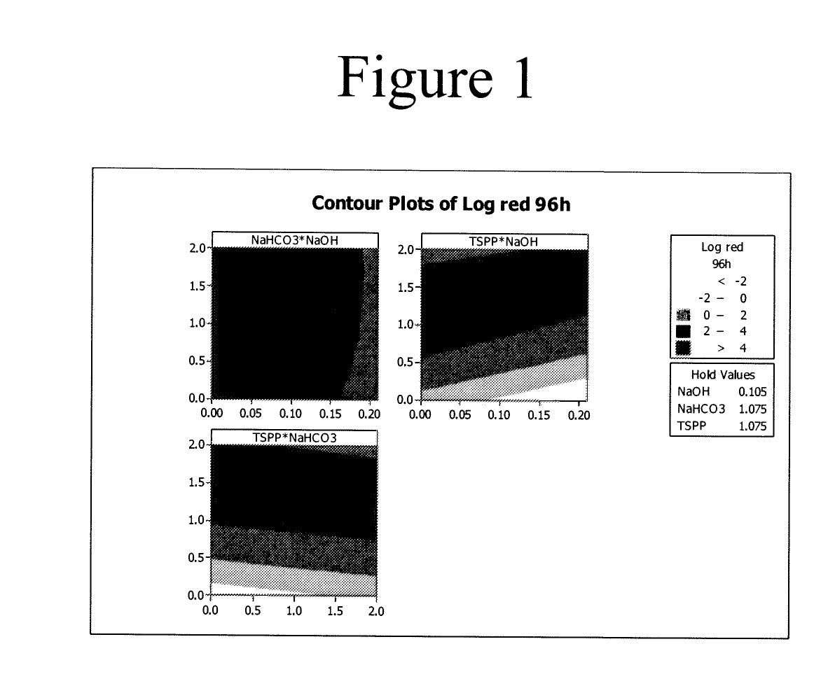

### DETAILED DESCRIPTION

As used herein, the term “oral composition” means the total composition that is delivered to the oral surfaces. The composition is further defined as a product which, during the normal course of usage, is not, the purposes of systemic administration of particular therapeutic agents, intentionally swallowed but is rather retained in the oral cavity for a time sufficient to contact substantially all of the dental surfaces and/or oral tissues for the purposes of oral activity. Examples of such compositions include, but are not limited to, toothpaste or a dentifrice, a mouthwash or a mouth rinse, a topical oral gel, a denture cleanser, and the like.

As used herein, the term “dentifrice” means paste, gel, or liquid formulations unless otherwise specified. The dentifrice composition can be in any desired form such as deep striped, surface striped, multi-layered, having the gel surrounding the paste, or any combination thereof. Alternatively the oral composition may be dual phase dispensed from a separated compartment dispenser.

As used herein, the term “precipitation agent(s)” means a compound or substance that is capable of precipitating out soluble alkaline earth metal ions, e.g. calcium ions (Ca²⁺) or strontium ions (Sr²⁺). Examples of precipitation agents include, but are not limited to, tetrasodium pyrophosphate and Na₂SiO₃.

As used herein, the terms “alkaline earth metal ion scavenging agent(s)” or “alkaline earth metal scavenging agents(s)” mean a compound or substance capable of decreasing the concentration of an alkaline earth metal ion (e.g., calcium ion [Ca²⁺] or strontium ion [Sr²⁺]) by increasing the concentration of common ions. Examples of alkaline earth metal ion scavenging agents include, but are not limited to, Na₂HPO₄ and NaHCO₃.

As used herein, the term “calcium ion scavenging agent(s)” means a compound or substance capable of decreasing the concentration of calcium ions (Ca²⁺) by increasing the concentration of common ions.

As used herein, the term “strontium ion scavenging agent(s)” means a compound or substance capable of decreasing the concentration of strontium ions (Sr²⁺) by increasing the concentration of common ions.

As used herein, the terms “high salt concentration” and “high concentration of salt” means a salt (e.g., sodium chloride) concentration of 50 g/l or greater.

Active and other ingredients useful herein may be categorized or described by their cosmetic and/or therapeutic benefit or their postulated mode of action. However, it is to be understood that the active and other ingredients useful herein can in some instances provide more than one cosmetic and/or therapeutic benefit or operate via more than one mechanism of action. Therefore, classifications are made for the sake of convenience and are not intended to limit an ingredient to the particularly stated application or the applications listed.

In some embodiments, the present invention provides compositions comprising an alkaline earth metal salt; benzyl alcohol; one or more precipitation agents; and one or more alkaline earth metal ion scavenging agents. In some embodiments, the alkaline earth metal salt is selected from a magnesium salt; a calcium salt; and a strontium salt. In some embodiments, the calcium salt is calcium carbonate. In some embodiments, the strontium salt is strontium chloride or strontium acetate.

In some embodiments, the present invention provides oral care compositions comprising: calcium carbonate; benzyl alcohol; one or more precipitation agents; and one or more alkaline earth metal ion scavenging agents. In some embodiments, at least one of said one or more precipitation agents is selected from sodium silicate and tetrasodium pyrophosphate. In some embodiments, at least one of said one or more precipitation agents is tetrasodium pyrophosphate.

In further embodiments, at least one of said one or more alkaline earth metal ion scavenging agents is selected from monosodium phosphate; disodium hydrogen phosphate; and sodium bicarbonate. In some embodiments, at least one of said one or more alkaline earth metal ion scavenging agents is sodium bicarbonate.

As used herein, the term “buffering system” means one or more ingredients which are able to maintain the composition within the desired pH range, to provide the optimal concentration of free alkaline earth metal ions. In some embodiments, the optimal concentration of free alkaline earth metal ions is dependent on the alkaline earth metal present in the composition. Some embodiments provide compositions comprising benzyl alcohol; an alkaline earth metal salt; and a buffering system. In some embodiments, the buffering system comprises sodium bicarbonate; tetrasodium pyrophosphate; and sodium hydroxide. In some embodiments, the buffering system comprises sodium bicarbonate and tetrasodium pyrophosphate. In other embodiments, the buffering system comprises sodium bicarbonate and sodium hydroxide. In some embodiments, the buffering system comprises tetrasodium pyrophosphate and sodium hydroxide.

In some embodiments, the compositions comprise from about 30% to about 50%, by weight, of an alkaline earth metal salt; from about 0.2% to about 0.5%, by weight, benzyl alcohol; from about 0.05% to about 2%, by weight, of one or more precipitation agents; and from about 0.05% to about 2%, by weight, of one or more alkaline earth metal ion scavenging agents.

In some embodiments, the compositions comprise from about 30% to about 50%, by weight, calcium carbonate; from about 0.2% to about 0.5%, by weight, benzyl alcohol; from about 0.05% to about 2%, by weight, of one or more precipitation agents; and from about 0.05% to about 2%, by weight, of one or more calcium ion scavenging agents.

In other embodiments, the present invention provides compositions comprising: from about 35% to about 45%, by weight, of an alkaline earth metal salt; from about 0.2% to about 0.5%, by weight, benzyl alcohol; from about 0.1% to about 1%, by weight, of one or more precipitation agents; and from about 0.1% to about 1%, by weight, of one or more alkaline earth metal ion scavenging agents.

Some embodiments provide oral care compositions comprising: from about 35% to about 45%, by weight, of an alkaline earth metal salt; about 0.5%, by weight, of a precipitation agent; about 0.5%, by weight, of an alkaline earth metal ion scavenging agent; and about 0.3%, by weight, benzyl alcohol.

In other embodiments, the present invention provides compositions comprising: from about 35% to about 45%, by weight, calcium carbonate; from about 0.2% to about 0.5%, by weight, benzyl alcohol; from about 0.1% to about 1%, by weight, of one or more precipitation agents; and from about 0.1% to about 1%, by weight, of one or more calcium ion scavenging agents. Some embodiments provide oral care compositions comprising: from about 35% to about 45%, by weight, calcium carbonate; about 0.5%, by weight, of a precipitation agent; about 0.5%, by weight, of a calcium ion scavenging agent; and about 0.3%, by weight, benzyl alcohol.

Some embodiments further comprise one or more pH modifying agents. In some embodiments, at least one of said one or more pH modifying agents is selected from the group consisting of: sodium hydroxide; potassium hydroxide; phosphoric acid; benzoic acid and citric acid. In other embodiments, at least one of said one or more pH modifying agents is sodium hydroxide. In some embodiments, the sodium hydroxide comprises from about 0.05% to about 0.2%, by weight, of the composition. Further embodiments provide compositions wherein the sodium hydroxide comprises about 0.1%, by weight, of the composition.

In some embodiments, the pH of the composition is from about 9 to about 10. In other embodiments, the pH of the composition is from about 9.2 to about 9.8. Still other embodiments provide compositions wherein the pH of the composition is from about 9.3 to about 9.6. In some embodiments, the pH of the composition is about 9.5.

In some embodiments, the alkaline earth metal salt comprises from about 5% to about 50%, by weight, of the composition. In other embodiments, the alkaline earth metal salt comprises from about 10% to about 40%, by weight, of the composition. In some embodiments, the alkaline earth metal salt comprises about 10%, by weight, of the composition. In some embodiments, the alkaline earth metal salt comprises about 5%, by weight, of the composition.

In some embodiments, the alkaline earth metal salt comprises about 37%, by weight, of the composition. In some embodiments, the calcium carbonate comprises about 37%, by weight, of the composition. In some embodiments, the alkaline earth metal salt comprises about 40%, by weight, of the composition. In some embodiments, the calcium carbonate comprises about 40%, by weight, of the composition.

Some embodiments of the present invention provide a bacteriostatic system comprising one or more precipitation agents; one or more alkaline earth metal ion scavenging agents; and benzyl alcohol. Some embodiments provide oral care compositions comprising a bacteriostatic system; and an orally acceptable carrier. In some embodiments, the orally acceptable carrier comprises an alkaline earth metal salt.

In some embodiments, the compositions comprise: calcium carbonate; and a bacteriostatic system comprising: one or more precipitation agents; one or more alkaline earth metal ion scavenging agents; and benzyl alcohol. In some embodiments, the oral care compositions comprise: strontium chloride; and a bacteriostatic system comprising: one or more precipitation agents; one or more alkaline earth metal ion scavenging agents; and benzyl alcohol. In some embodiments, the oral care compositions comprise: strontium acetate; and a bacteriostatic system comprising: one or more precipitation agents; one or more alkaline earth metal ion scavenging agents; and benzyl alcohol.

In some embodiments, the bacteriostatic system comprises: from about 0.05% to about 2%, by weight, tetrasodium pyrophosphate; from about 0.05% to about 2%, by weight, sodium bicarbonate; from about 0.2% to about 0.5%, by weight, benzyl alcohol; and from about 0.05% to about 0.2%, by weight, of sodium hydroxide.

Some embodiments provide compositions comprising: from about 0.1% to about 1%, by weight, tetrasodium pyrophosphate; from about 0.1% to about 1%, by weight, sodium bicarbonate; about 0.3%, by weight, benzyl alcohol; and about 0.1%, by weight, sodium hydroxide. Further embodiments provide compositions comprising: about 0.5%, by weight, tetrasodium pyrophosphate; and about 0.5%, by weight, sodium bicarbonate.

In some embodiments, the concentration of HPO₄²⁻ or CO₃²⁻ is maximized in order to minimize the alkaline earth metal ion concentration. In some embodiments, the concentration of HPO₄²⁻ or CO₃²⁻ is maximized in order to minimize the Ca²⁺ concentration. In some embodiments, the concentration of HPO₄²⁻ or CO₃²⁻ is maximized in order to minimize the Sr²⁺ concentration.

Some embodiments of the present invention provide compositions which further comprise a humectant. In some embodiments, the humectant is selected from the group consisting of: sorbitol; glycerin; polyethylene glycol; propylene glycol; and other edible polyhydric alcohols. In various embodiments, humectants are operable to prevent hardening of paste or gel compositions upon exposure to air. In some embodiments humectants also function as sweeteners.

Some embodiments of the present invention provide methods of inhibiting a disease, disorder or condition of the oral cavity comprising contacting an oral cavity surface with any of the compositions described herein. Some embodiments of the present invention provide methods of preventing a disease, disorder or condition of the oral cavity comprising contacting an oral cavity surface with any of the compositions described herein. Some embodiments of the present invention provide methods of treating a disease, disorder or condition of the oral cavity comprising contacting an oral cavity surface with any of the compositions described herein. In some embodiments, the disease, disorder or condition of the oral cavity is an inflammatory disease, disorder or condition. In some embodiments, the disease, disorder or condition of the oral cavity is selected from the group consisting of gingivitis; periodontitis; and halitosis. In some embodiments, the present invention provides methods of whitening a tooth surface comprising contacting a tooth surface with any of the compositions described herein.

In some embodiments, the compositions described herein prevent the growth of bacteria resistant to high salt concentrations. In some embodiments, the bacteria resistant to high salt concentrations are halophilic bacteria. In some embodiments, the halophilic bacteria are from the halomonas species. Halophilic bacteria are characterized by their ability to grow in media containing concentrations of sodium chloride that usually completely inhibit the multiplication of non-halophilic species. Robinson et al., J. Bacteriol. 1952 July; 64(1):69-77.

In some embodiments, compositions of the present invention further comprise safe and effective levels of one or more additional components. Such materials are well known and are readily chosen by those skilled in the art based on the oral care, physical and aesthetic properties desired for the compositions being prepared. Examples of such materials include, but are not limited to fats, solvents, waxes, emulsifiers, softeners, bulking agents, cationic materials, buffers, whitening agents, alkali metal bicarbonate salts, thickening agents, water, surfactants, flavoring agents, coloring agents, and mixtures thereof.

Some embodiments comprise suitable abrasives such as silica, for example in the form of silica gel, hydrated silica or precipitated silica, alumina, and insoluble phosphates.

Polishing agents such as silica, calcined alumina, sodium bicarbonate, calcium carbonate, dicalcium phosphate and calcium pyrophosphate may be included in the base dentifrice compositions used in the practice of the present invention. Visually clear dentifrice compositions are obtained by using polishing agents such as collodial silica, such as those sold under the trade designation Zeodent 115 available from the Huber Corporation or alkali metal aluminosilicate complexes (that is, silica containing alumina combined in its matrix) which have refractive indices close to the refractive indices of gelling agent-liquid (including water and/or humectant) systems used in dentifrice compositions. The polishing agent is generally present in the base dentifrice composition in weight concentrations of about 3% to about 50% by weight.

Some embodiments provide compositions further comprising an oral care active selected from the group consisting of an anti-calculus agent; an anti-plaque agent; a fluoride ion source; a desensitizing agent; an oral malodor control agent; a H₂ antagonist; and mixtures thereof. Optional desensitizing agents include potassium citrate, potassium chloride, potassium tartrate, potassium bicarbonate, potassium oxalate, potassium nitrate, strontium salts, and mixtures thereof.

In some embodiments, the compositions of the present invention further comprise an amino acid. In some embodiments, the amino acid is present in a desensitizing effective amount. In some embodiments, the amino acid comprises from about 0.01% to about 10%, by weight, of the composition. In some embodiments, the amino acid comprises from about 0.1% to about 7%, by weight, of the composition. In some embodiments, the amino acid comprises from about 0.5% to about 5%, by weight, of the composition. In some embodiments, the amino acid comprises from about 1% to about 4%, by weight, of the composition. In some embodiments, the amino acid comprises from about 2% to about 3%, by weight, of the composition. In some embodiments, the amino acid comprises about 2.5%, by weight, of the composition. In some embodiments, the amino acid comprises arginine. In some embodiments, the amino acid comprises L-arginine. In some embodiments, the amino acid comprises L-arginine bicarbonate. In some embodiments, L-arginine bicarbonate comprises about 2.5%, by weight, of the composition.

In some embodiments, the anti-calculus agent is selected from: a phosphate, a pyrophosphate; a polyphosphate; a phosphonate; a polyphosphonate; and mixtures thereof. In some embodiments, the pyrophosphate is selected from: a dialkali metal pyrophosphate salt; a tetra-alkali metal pyrophosphate salt; and mixtures thereof in their unhydrated as well as hydrated forms. Disodium dihydrogen pyrophosphate (Na₂H₂P₂O₇), tetrasodium pyrophosphate (Na₄P₂O₇), and tetrapotassium pyrophosphate (K₄P₂O₇) and mixtures thereof. Pyrophosphate salts suitable for use in the compositions of the present invention are described in more detail in Kirk and Othmer, Encyclopedia of Chemical Technology, 3^{rd} Edition, Vol. 17, Wiley Interscience Publishers (1982).

Additional anti-calculus agents include polyacrylates and other polycarboxylates such as those disclosed in U.S. Pat. No. 3,429,963 and U.S. Pat. No. 4,304,766; and U.S. Pat. No. 4,661,341; polyepoxysuccinates such as those disclosed in U.S. Pat. No. 4,846,650; ethylenediaminetetraacetic acid as disclosed in British Patent No. 490,384; nitrilotriacetic acid and related compounds as disclosed in U.S. Pat. No. 3,678,154; polyphosphonates as disclosed in U.S. Pat. No. 3,737,533; U.S. Pat. No. 3,988,443 and U.S. Pat. No. 4,877,603. Anticalculus phosphates include potassium and sodium pyrophosphates; sodium tripolyphosphate; diphosphonates such as ethane-1-hydroxy-1,1-diphosphonate, 1-azacycloheptane-1,1-diphosphonate, and linear alkyl diphosphonates; linear carboxylic acids; and sodium zinc citrate and other soluble zinc salts.

A wide variety of fluoride ion yielding materials can be employed as sources of soluble fluoride in the compositions of the present invention. Examples of suitable fluoride ion yielding materials can be found in U.S. Pat. Nos. 3,535,421 and 3,678,154. In some embodiments, the fluoride ion yielding material is selected from: sodium fluoride; potassium fluoride; stannous fluoride; ammonium fluoride; sodium monofluorophosphate; and mixtures thereof.

In some embodiments, the compositions of the present invention further comprise an oral malodor control agent. Such agents may include, but are not limited to, magnesium mono-potassium phthalate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenifine; delmopinol; octapinol; and other piperidine derivatives; nicin preparations; zinc/stannous ion agents; antibiotics such as augmentin, amoxicillin, tetracycline, doxycycline, minocycline, and metronidazole; and analogues and salts of the above; methyl salicyclate; and mixtures of all of the above.

Compositions of the present invention may also comprise surfactants, commonly referred to as sudsing agents. Suitable surfactants are those which are reasonably stable and foam throughout a wide pH range. The surfactant may be anionic, amphoteric, zwitterionic, cationic, or mixtures thereof.

Anionic surfactants useful herein include the water soluble salts of alkyl sulphates having from about 8 to about 20 carbon atoms in the alkyl radical (eg sodium alkyl sulphate) and the water soluble salts of sulphonates monoglycerides of fatty acids having from about 8 to about 20 carbon atoms. Sodium lauryl sulphate and socium coconut monoglyceride sulphonates are examples of anionic surfactants of this type. Many suitable anionic surfactants are disclosed in U.S. Pat. No. 3,959,458.

Nonionic surfactants which can be used in the compositions of the present invention can be broadly designed as compounds produced by the condensation of alkylene oxide groups (hydrophilic in nature) with an organic hydrophobic compound which may be aliphatic or alkyl-aromatic in nature.

The amphoteric surfactants useful in the present invention can be broadly described as derivatives of aliphatic secondary and tertiary amines in which the aliphatic radical can be straight chain or branched and wherein one of the aliphatic substituents contains from about 8 to about 18 carbon atoms and one contains an anionic water solubilising group eg carboxylate, sulphonate, suphate, phosphate or phosphonate. Many of these suitable non-ionic and amphoteric surfactants are disclosed in U.S. Pat. No. 4,051,234.

Other optional additives include antimicrobial (e.g., antibacterial) agents. Any orally acceptable antimicrobial agent can be used, including halogentated diphenylethers such as triclosan(5-chloro-2-(2,4-dichlorophenoxy)phenol); 8-hydroxyquinoline and salts thereof, zinc and stannous ion sources such as zinc citrate, zinc sulphate, zinc glycinate, sodium zinc citrate and stannous pyrophosphate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), combinations of CPC with zinc and/or enzymes, tetradecylpyridinium chloride, and N-tetradecyl-4-ethylpyridinium chloride,); bisguanides, such as chlorhexidine digluconate, hexetidine, octenidine, alexidine; halogenated bisphenolic compounds, such as 2,2′ methylenebis-(4-chloro-6-bromophenol); benzalkonium chloride; salicylanilide, domiphen bromide; iodine; sulfonamides; bisbiguanides; phenolics; piperidino derivatives such as delmopinol and octapinol; magnolia extract; grapeseed extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin and clindamycin; and mixtures thereof. A further illustrative list of useful antibacterial agents is provided in U.S. Pat. No. 5,776,435.

Antioxidants are another class of optional additives. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

Also optional, a saliva stimulating agent, useful for example in amelioration of dry mouth may be included. Any orally acceptable saliva stimulating agent can be used, including without limitation food acids such as citric, lactic, malic, succinic, ascorbic, adipic, fumaric, and tartaric acids, and mixtures thereof. One or more saliva stimulating agents are optionally present in a saliva stimulating effective amount.

Optional breath freshening agents may be provided. Any orally acceptable breath freshening agent can be used, including without limitation zinc salts such as zinc gluconate, zinc citrate and zinc chlorite, alpha-ionone and mixtures thereof. One or more breath freshening agents are optionally present in a breath freshening effective total amount.

In various embodiments, the compositions of this invention comprise a peroxide whitening agent, comprising a peroxide compound. A peroxide compound is an oxidizing compound comprising a bivalent oxygen-oxygen group. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof. In one embodiment, the peroxide compound comprises hydrogen peroxide. In one embodiment, the peroxide compound consists essentially of hydrogen peroxide.

In some embodiments a non-peroxide whitening agent may be provided. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite. One or more whitening agents are optionally present in a tooth-whitening effective amount.

Flavoring agents for incorporation in the compositions may include natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. These flavor agents can be used individually or in admixture. Commonly used flavors include mints such as peppermint, artificial vanilla, cinnamon derivatives, and various fruit flavors, whether employed individually or in admixture. Generally, any flavoring or food additive, such as those described in Chemicals Used in Food Processing, publication 1274 by the National Academy of Sciences, pages 63-258, may be used.

The compositions of the present invention may also comprise colorants. In some embodiments, the colorant can be a dye or a pigment. Dyes suitable for use in compositions of the present invention may be food color additives presently certified under the Food Drug & Cosmetic Act for use in food and ingested drugs, including dyes such as FD&C Red No. 3 (sodium salt of tetraiodofluorescein), Food Red 17, disodium salt of 6-hydroxy-5-{(2-methoxy-5-methyl-4-sulphophenyl)azo}-2-n-aphthalenesulfonic acid, Food Yellow 13, sodium salt of a mixture of the mono and disulphonic acids of quinophtalone or 2-(2-quinolyl)indanedione, FD&C Yellow No. 5 (sodium salt of 4-p-sulfophenylazo-1-p-sul-fophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-(N-ethyl-N-p-sulfobenzyl)-.DELTA.-3,5-cycl-ohexadienimine], FD&C Blue No. 1 (disodium salt of dibenzyldiethyl-diamino-triphenylcarbinol trisulfonic acid anhydrite), FD&C Blue No. 2 (sodium salt of disulfonic acid of indigotin) and mixtures thereof in various proportions.

The invention is further described in the following examples. The examples are merely illustrative and do not in any way limit the scope of the invention as described and claimed.

### EXAMPLES

### Example 1

Table 1 (below) describes exemplary compositions (Formulae I-VI) of the present invention. CI-CIII serve as comparative examples to Formulae I-IV, while CIV serves as a comparative example to Formulae V and VI.

TABLE 1

| Ingredient | I | II | III | IV | CI | CII | CIII | V | VI | CIV |
|---|---|---|---|---|---|---|---|---|---|---|
| Na Saccharin | 0.2 | 0.2 | 0.2 | 0.2 | 0.2 | 0.2 | 0.2 | 0.3 | 0.3 | 0.3 |
| Carboxymethylcellulose | 0.8 | 0.8 | 0.8 | 0.8 | 0.8 | 0.8 | 0.8 | 0.9 | 0.9 | 0.9 |
| Sodium | 1.1 | 1.1 | 1.1 | 1.1 | 1.1 | 1.1 | 1.1 | 1.1 | 1.1 | 1.1 |
| Monofluorophosphate | | | | | | | | | | |
| Vanillin Replacement | 0.06 | 0.06 | 0.06 | 0.06 | 0.06 | 0.06 | 0.06 | — | — | — |
| Benzyl Alcohol | 0.3 | 0.3 | 0.3 | 0.3 | — | — | — | 0.3 | 0.3 | — |
| NaOH | — | 0.1 | — | 0.1 | — | — | 0.1 | — | 0.1 | — |
| NaSiO₃ | 1 | — | 0.4 | — | 1 | 1 | — | 1 | — | 0.8 |
| NaHCO₃ | 0.5 | 0.5 | 0.5 | 1 | 0.5 | 0.5 | 0.5 | 0.5 | 0.5 | 0.5 |
| Sodium Lauryl Sulfate | 4.6 | 4.6 | 4.6 | 4.6 | 4.6 | 4.6 | 4.6 | 5 | 5 | 5 |
| Flavor | 1.1 | 1.1 | 1.1 | 1.1 | 1.1 | 1.1 | 1.1 | 1 | 1 | 1 |
| Tetrasodium | — | 0.5 | 0.5 | 1 | — | — | 0.5 | — | 0.5 | — |
| Pyrophosphate | | | | | | | | | | |
| Sorbitol | 23 | 23 | 23 | 23 | 23 | 23 | 23 | 23 | 23 | 23 |
| Parabens | — | — | — | — | 0.12 | 0.19 | 0.19 | — | — | 0.12 |
| Calcium Carbonate | 40 | 40 | 40 | 40 | 40 | 40 | 40 | 37 | 37 | 37 |
| Xanthan Gum | — | — | — | — | — | — | — | 0.21 | 0.21 | 0.21 |
| Pigment Blue #15 | — | — | — | — | — | — | — | 0.01 | 0.01 | 0.01 |
| Water | 27.3 | 27.7 | 27.4 | 26.7 | 27.5 | 27.4 | 27.8 | 29.6 | 30 | 30 |

### Example 2

### Micro Robustness Test or Micro Challenge Test

The Micro Robustness Test or Micro Challenge Test is used to screen products to assess the formulas robustness. It is a quantitative measure of the formula's ability to withstand microbial insult, both at the plant and in the hands of the consumers, and encompasses the rate of kill of the bacterial inoculum as well as the total kill level. This quantitative measure is defined as the Area Under the Curve (AUC).

Products sample are challenged with a selected inoculum pool. At selected time intervals, the inoculated test material is sampled. Dilutions and platings are performed to recover the surviving organisms. The log differences in the bacterial count (Log reduction) between the product and the inoculum control is calculated over time to determine the AUC.

The results of the Micro Robustness Test, presented in Log red., indicate the effectiveness of a preservative or bacteriostatic system—the greater the Log red. value, the more effective the preservative.

Table 2 (below) compares the micro-robustness of compositions of the present invention versus Comparative Examples I-IV. The data described therein, demonstrates that compositions of the present invention provide consistent micro-robustness, while the compositions of the comparative examples do not. Specifically, Formulae I-VI all provide a log reduction in bacterial growth over 96 hrs, while only one of the comparative examples was able to resist bacterial growth to the same extent over the same period of time. The micro count time was extended to 96 hours to capture effectiveness against certain microorganisms which develop more slowly.

TABLE 2

| | Log Red | Log Red |
|---|---|---|
| Formula | 4 hrs | 96 hrs |
| I | 6.3 | 6.3 |
| II | 6.3 | 6.3 |
| III | 6.3 | 6.3 |
| IV | 6.3 | 6.3 |
| V | 6.3 | 6.3 |
| VI | 6.3 | 6.3 |
| CI | 3.2 | 1 |
| CII | 4.9 | 2.1 |
| CIII | 6.3 | 6.3 |
| CIV | 3.2 | — |

### Example 3

Compositions of the present invention can be prepared according to methods known in the art. By way of example, and not limitation, a method of preparation is provided herein.

Part I: Gel Phase

Weigh appropriate quantities of water, sorbitol, sodium monofluorophosphate, sodium saccharin, tetrasodium pyrophosphate, carboxymethyl cellulose, and sodium hydroxide and transfer to a gel mixer. Mix for about 15 minutes. Transfer resultant gel product to the main mixer.

Part II: Main Mixer

Weigh appropriate quantities of precipitated calcium carbonate, sodium lauryl sulfate, and benzyl alcohol and transfer to the main mixer. Mix for about 5 minutes. Allow product to cool for time sufficient to reach temperature below about 46° C. Weigh appropriate quantities of flavor and vanillin replacement, and add to main mixer. Allow product to cool for time sufficient to reach temperature below about 46° C. Mix under full vacuum for about 15 minutes. Collect finished product from main mixer.

Each of the patents, patent applications and printed publications (including books) mentioned in this document are hereby incorporated by reference in their entirety.

As those skilled in the art will appreciate, numerous changes and modifications may be made to the embodiments described herein without departing from the spirit of the invention. It is intended that all such variations fall within the scope of the appended claims.

1. An oral care composition comprising:
calcium carbonate;
benzyl alcohol;
one or more precipitation agents; and
one or more calcium ion scavenging agents.

2. The composition of claim 1, wherein at least one of said one or more precipitation agents is selected from sodium silicate and tetrasodium pyrophosphate.

3. The composition of claim 1, wherein at least one of said one or more calcium ion scavenging agents is selected from monosodium phosphate; disodium hydrogen phosphate and sodium bicarbonate.

4. The composition of claim 1, comprising:
from about 30% to about 50%, by weight, calcium carbonate;
from about 0.2% to about 0.5%, by weight, benzyl alcohol;
from about 0.05% to about 2%, by weight, of one or more precipitation agents selected from sodium silicate and tetrasodium pyrophosphate; and
from about 0.05% to about 2%, by weight, of one or more calcium ion scavenging agents selected from monosodium phosphate; disodium hydrogen phosphate and sodium bicarbonate.

5. The composition of claim 4, comprising:
from about 35% to about 45%, by weight, calcium carbonate;
from about 0.2% to about 0.5%, by weight, benzyl alcohol;
from about 0.1% to about 1%, by weight, of one or more precipitation agents selected from sodium silicate and tetrasodium pyrophosphate; and
from about 0.1% to about 1%, by weight, of one or more calcium ion scavenging agents selected from monosodium phosphate; disodium hydrogen phosphate and sodium bicarbonate.

6. The composition of claim 1, further comprising one or more pH modifying agents selected from the group consisting of: sodium hydroxide; potassium hydroxide; phosphoric acid; benzoic acid and citric acid and wherein the pH of the composition is from about 9.3 to about 9.6.

7-13. (canceled)

14. An oral care composition of claim 5 comprising:
from about 35% to about 45%, by weight, calcium carbonate;
about 0.3%, by weight, benzyl alcohol;
about 0.5%, by weight, of a precipitation agent selected from sodium silicate and tetrasodium pyrophosphate; and
about 0.5%, by weight, of a calcium ion scavenging agent selected from monosodium phosphate; disodium hydrogen phosphate and sodium bicarbonate.

15. The composition of claim 14, wherein said precipitation agent is tetrasodium pyrophosphate and said calcium ion scavenging agent is sodium bicarbonate.

16. (canceled)

17. The composition of claim 14, further comprising one or more pH modifying agents selected from the group consisting of: sodium hydroxide; potassium hydroxide; phosphoric acid; benzoic acid and citric acid and wherein the pH of the composition is from about 9.3 to about 9.6.

18-36. (canceled)

37. The composition of claim 4, further comprising L-arginine bicarbonate.

38. The composition of claim 14, further comprising L-arginine bicarbonate.

39. (canceled)
